(19) United States
(12) Patent Application Publication
(10) Pub. No.: US 2004/0260124 A1
(43) Pub. Date: Dec. 23, 2004

(54) Tertiary amine and method for producing the same

(76) Inventors: Toshiaki Murai, Gifu-shi (JP); Masahiro Murakami, Kyoto-shi (JP); Yuichiro Mutoh, Gifu-ken (JP); Yukiyasu Ohta, Iwata-shi (JP)

Correspondence Address: FISH & RICHARDSON PC, 225 FRANKLIN ST, BOSTON, MA, 02110, US

(21) Appl. No.: 10/761,010

(22) Filed: Jan. 20, 2004

(30) Foreign Application Priority Data
Jun. 20, 2003 (JP) 2003-177181

(51) Int. Cl. C07F 7/04; C07C 239/08

(52) U.S. Cl. 564/300; 556/413; 564/301

(57) ABSTRACT

The present invention relates to a method for easily producing a tertiary amine with high yield. A tertiary amine represented by general formula (1) is produced by adding a metal-containing reagent represented by general formula (6) into a reaction system consisting of thioamide represented by general formula (4), a methylating agent represented by general formula (5) and a solvent, and then adding thereto a Grignard reagent represented by general formula (7).

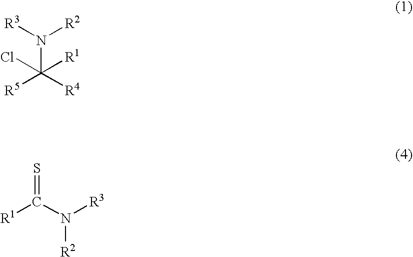

CH₃—X (5)

R⁸-M¹ (6)

R⁷-M² (7)

### BACKGROUND OF THE INVENTION

The present invention relates to novel tertiary amines used for various synthetic materials, various chemical products, pharmaceutical preparations, agricultural chemicals and others, and a method for easily producing a tertiary amine with high yield.

As described in Japanese Patent Laid-Open No. 58-69845, tertiary amines, such as dimethylamino-8-(4-chlorophenyl)-prop-1-yne, are conventionally used as metal corrosion inhibitors. Tertiary amine is produced by the reaction among secondary amine, such as dibutylamine, aldehyde, such as 2-chlorobenzaldehyde, and acetylene. Since secondary amine, aldehyde, and acetylene hardly react at room temperature, synthetic reaction of tertiary amine is carried out using a catalyst, such as copper, under a relatively high pressure of approximately 20 atmosphere at a relatively high temperature of approximately 95° C. Accordingly, the conventional production method of tertiary amine is complicated.

A method for synthesizing a novel tertiary amine from a compound with delocalized electrons having sulfur atom have been desired. It is expected that such novel tertiary amine will have a new physiological activity that differs from those of the known tertiary amines, and it is also expected that the novel tertiary amine can be used as a base compound for various synthetic materials.

### SUMMARY OF THE INVENTION

It is an object of the present invention to provide a method for easily producing a tertiary amine with high yield, and novel tertiary amines.

To achieve the above object, the present invention provides a tertiary amine represented by the following general formula (1):

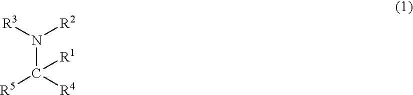

wherein R¹ represents hydrogen atom, alkyl group or aryl group; R² and R³ each represent alkyl group or allyl group; R⁴ represents alkyl group, aryl group or allyl group; R⁵ represents alkynyl group, aryl group or alkyl group; and wherein when R⁵ is aryl or alkyl group, R¹, R⁴ and R⁵ are different from one another.

A further aspect of the present invention is a method for producing a tertiary amine represented by the following general formula (3):

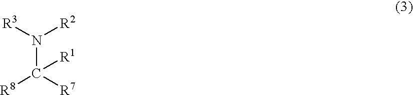

wherein R¹ represents hydrogen atom, alkyl group or aryl group; R² and R³ each represent alkyl group or allyl group; R⁷ represents alkyl group, aryl group, allyl group, vinyl group or alkynyl group; and R⁸ represents alkynyl group, aryl group or alkyl group. The method includes adding thioamide represented by general formula (4) and a methylating agent represented by general formula (5) into a solvent; adding thereto a metal-containing reagent represented by general formula (6); and adding thereto a Grignard reagent represented by general formula (7),

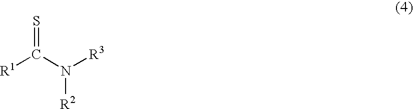

wherein R¹ represents hydrogen atom, alkyl group or aryl group, and R² and R³ each represent alkyl group or allyl group,

CH₃—X (5)

wherein X represents perfluoroalkyl sulfoxyl group,

R⁸-M¹ (6)

wherein R⁸ represents alkynyl group, aryl group or alkyl group, and M¹ represents an alkali metal atom, and

R⁷-M² (7)

wherein R⁷ represents alkyl group, aryl group, allyl group, vinyl group or alkynyl group, and M² represents MgCl, MgBr or MgI.

Other aspects and advantages of the present invention will become apparent from the following description, taken in conjunction with the accompanying drawings, illustrating by way of example the principles of the invention.

### DETAILED DESCRIPTION OF THE PREFERRED EMBODIMENTS

An embodiment of the present invention will be described below.

The present embodiment relates to a tertiary amine represented by the following general formula (1), which has physiological activity and is used for pharmaceutical preparations, agricultural chemicals, and chemical products:

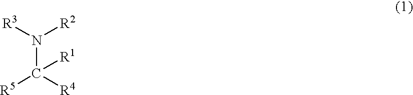

wherein R¹ represents hydrogen atom, alkyl group or aryl group; R² and R³ each represent alkyl group or allyl group; R⁴ represents alkyl group, aryl group or allyl group; R⁵ represents alkynyl group, aryl group or alkyl group; and when R⁵ is aryl or alkyl group, R¹, R⁴ and R⁵ are different from one another.

R¹ in the general formula (1) may include alkyl group, such as methyl group, propyl group, isopropyl group, butyl group, and n-butyl group; and aryl group, such as phenyl group and bromophenyl group.

R² and R³ may include alkyl group, such as methyl group.

R⁴ may include alkyl group, such as ethyl group, butyl group, n-butyl group, or trimethylsilylmethyl group; and aryl group, such as phenyl group.

R⁵ may include: alkyl group, such as ethyl group, butyl group, and n-butyl group; and aryl group, such as phenyl group.

A tertiary amine represented by the general formula (1) in which R⁵ represents alkynyl group, that is, propargylamine represented by general formula (2), can be produced at high efficiency. Propargylamines wherein, in the general formula (2), R¹ represents hydrogen atom, alkyl group or aryl group, R² and R³ each represent alkyl group or allyl group, R⁴ represents alkyl group, aryl group or allyl group, and R⁶ represents dialkoxyalkyl group; or propargylamines wherein, in the same above formula, R¹ represents hydrogen atom, alkyl group or aryl group, R² and R³ each represent alkyl group or allyl group, R⁴ represents alkyl group or allyl group, and R⁶ represents silyl group or aryl group, can be produced at higher efficiency.

Examples of alkyl group having 2 or more carbon atoms represented by R⁶ in the general formula (2) include dialkoxyalkyl group, such as isopropenyl group and diethoxymethyl group. An aryl group represented by R⁶ may include phenyl group. A silyl group represented by R⁶ may include trimethylsilyl group.

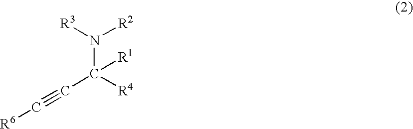

wherein R¹ represents hydrogen atom, alkyl group or aryl group; R² and R³ each represent alkyl group or allyl group; R⁴ represents alkyl group, aryl group or allyl group; and R⁶ represents alkyl group, aryl group, silyl group, vinyl group or formyl group having 2 or more carbon atoms.

The tertiary amine represented by the general formula (1) or (2) can be produced at much higher efficiency, when it is one selected from a group consisting of: N,N-dimethyl-1-phenyl-1-heptyn-3-amine (a case where R¹ is hydrogen atom, R² and R³ are methyl group, R⁴ is n-butyl group, and R⁶ is phenyl group); N,N-dimethyl-6-(trimethylsilyl)-1-hexen-5-yn-3-amine (a case where R¹ is hydrogen atom, R² and R³ are methyl group, R⁴ is allyl group, and R⁶ is trimethylsilyl group); N,N-dimethyl-α-(3-methyl-3-buten-1-ynyl)-benzenemethanamine (a case where R¹ is hydrogen atom, R² and R³ are methyl group, R⁴ is phenyl group, and R⁶ is isopropenyl group); N,N-dimethyl-α-(3,3-diethoxy-1-propynyl)-benzenemethanamine (a case where R¹ is hydrogen atom, R² and R³ are methyl group, R⁴ is phenyl group, and R⁶ is diethoxymethyl group); N,N-(di-2-propenyl)-α-(phenylethynyl)-benzenemethanamine (a case where R¹ is hydrogen atom, R² and R³ are allyl group, and R⁴ and R⁶ are phenyl group); N,N-dimethyl-α-(4-bromophenyl)-α-ethyl-benzenemethanamine (a case where R¹ is 4-bromophenyl group, R² and R³ are methyl group, R⁴ is ethyl group, and R⁵ is phenyl group); N,N-dimethyl-α-butyl-α-2-propenyl-benzenemethanamine (a case where R¹ is phenyl group, R² and R³ are methyl group, R⁴ is allyl group, and R⁵ is n-butyl group); N,N-dimethyl-α-methyl-α-[(trimethylsilyl)ethynyl]-benzenemethanamine (a case where R¹, R² and R³ are methyl group, R⁴ is phenyl group, and R⁶ is trimethylsilyl group); N,N-dimethyl-4-(1-methylethyl)-6-(trimethylsilyl)-1-hexen-5-yn-4-amine (a case where R¹ is isopropyl group, R²and R³ are methyl group, R⁴ is allyl group, and R⁶ is trimethylsilyl group); N,N-dimethyl-α-ethyl-α-[(trimethylsilyl)ethynyl]-benzenemethanamine (a case where R¹ is phenyl group, R² and R³ are methyl group, R⁴ is ethyl group, and R⁶ is trimethylsilyl group); N,N-dimethyl-α-(2-formylethynyl)-α-[(1-trimethylsilyl)methyl]-benzenemethanamine (a case where R¹ is phenyl group, R² and R³ are methyl group, R⁴ is trimethylsilylmethyl group, and R⁶ is formyl group); and N,N-dimethyl-α-(3-methyl-3-buten-1-ynyl)-α-(2-propenyl)-4-bromobenzenemethanamine (a case where R¹ is 4-bromophenyl group, R² and R³ are methyl group, R⁴ is allyl group, and R⁶ is isopropenyl group).

Next, a method for producing a tertiary amine represented by general formula (3) will be described. The tertiary amine represented by general formula (3) includes the tertiary amine represented by the general formula (1).

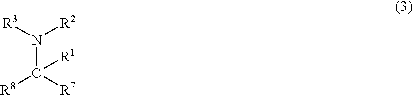

wherein R¹ represents hydrogen atom, alkyl group or aryl group; R² and R³ each represent alkyl group or allyl group; R⁷ represents alkyl group, aryl group, allyl group, vinyl group or alkynyl group; and R⁸ represents alkynyl group, aryl group or alkyl group.

For the general formula (3), examples of alkyl group include ethyl group, methyl group, propyl group, isopropyl group, butyl group, n-butyl group, and trimethylsilylmethyl group. Examples of aryl group include phenyl group and 4-bromophenyl group. Examples of alkynyl group include ethynyl group. In order to increase production efficiency, the tertiary amine represented by the general formula (3) is preferably a tertiary amine represented by general formula (9). For the general formula (9), examples of alkyl group having 2 or more carbon atoms include butyl group, n-butyl group and isopropenyl group, and dialkoxyalkyl group, such as diethoxymethyl group.

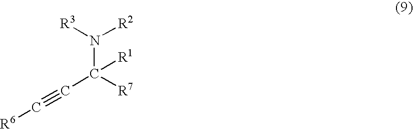

wherein R¹ represents hydrogen atom, alkyl group or aryl group; R² and R³ each represent alkyl group or allyl group; R⁷ represents alkyl group, aryl group, allyl group, vinyl group or alkynyl group; and R⁶ represents alkyl group, aryl group, silyl group, vinyl group or formyl group having 2 or more carbon atoms.

When the tertiary amine represented by the general formula (3) is produced, first, a thioamide represented by general formula (4) and an agent represented by general formula (5) are added into a solvent. Thereafter, a metal-containing reagent represented by general formula (6) is added to the reaction solution, and a Grignard reagent represented by general formula (7) is further added thereto. Thus, the components react as shown in reaction formula (10), so that the tertiary amine represented by the general formula (3) can be produced. It is noted that by-products are not shown in reaction formula (10).

In this case, the equivalent ratio of thioamide : methylating agent: metal-containing reagent: Grignard reagent is preferably within the range of 1:1:(1.2 to 1.5):(1.5 to 10). If the ratio of each component is less than the above range, the reaction does not sufficiently progress. In contrast, if the ratio of each component exceeds the above range, the reaction further insufficiently progresses, and thus, it is uneconomical.

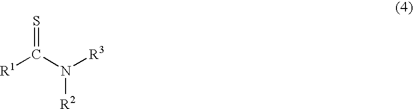

wherein R¹ represents hydrogen atom, alkyl group or aryl group, and R² and R³ each represent alkyl group or allyl group,

CH₃—X (5)

wherein X represents perfluoroalkyl sulfoxyl group,

R⁸-M¹ (6)

wherein R⁸ represents alkynyl group, aryl group or alkyl group, and M¹ represents an alkali metal atom, and

R⁷-M² (7)

wherein R⁷ represents alkyl group, aryl group, allyl group, vinyl group or alkynyl group, and M² represents MgCl, MgBr or MgI.

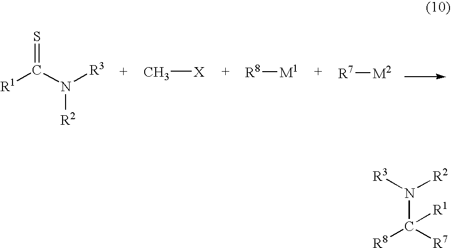

In the reaction formula (10), first, thioamide reacts with a methylating agent in a solvent, so as to generate a reaction intermediate. This reaction intermediate reacts with a metal-containing reagent, and then with a Grignard reagent, so as to generate a tertiary amine. This is to say, the reaction intermediate between thioamide and the methylating agent undergoes an addition reaction with the metal-containing reagent, so as to generate a first addition product. Thereafter, the first addition product undergoes an addition reaction with the Grignard reagent, so as to generate a second addition product (tertiary amine). Accordingly, the order of adding thioamide, a methylating agent, a metal-containing reagent and a Grignard reagent is important in the present embodiment.

If thioamide and the metal-containing reagent are added into a solvent, the methylating agent is then added thereto, and the Grignard reagent is then added thereto, the thioamide does not quickly react with the metal-containing reagent, but instead, the metal-containing reagent reacts with the methylating agent. Consequently, the production efficiency of the tertiary amine decreases. If the methylating agent and the metal-containing reagent are added into a solvent, the thioamide is then added thereto, and the Grignard reagent is then added thereto, the methylating agent reacts with the metal-containing reagent before addition of thioamide, and thereby a reaction intermediate necessary to obtain the tertiary amine cannot be generated, so that the tertiary amine cannot be generated.

Accordingly, it is necessary that thioamide and a methylating agent are first added into a solvent, that a metal-containing reagent is added thereto, and that a Grignard reagent is then added thereto. Since thioamide, the methylating agent, the metal-containing reagent and the Grignard reagent have a high reactivity, the reaction as shown in the reaction formula (10) progresses without using catalysts, so that the yield of the tertiary amine can be increased up to 95% and that the purity of the tertiary amine can be increased to 99% or more.

In the general formula (5), the perfluroalkyl sulfoxyl group is represented by general formula (11). Methyl triflate represented by general formula (12) is preferable because it is easily acquired and has a high reactivity with thioamide. In the general formula (6), lithium atom (Li), sodium atom (Na) or potassium atom (K) is preferable as M¹ because these atoms have a high reactivity with the reaction intermediate.

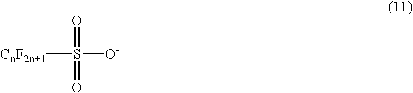

wherein n represents an integer between 1 and 8.

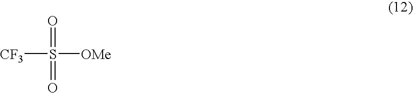

wherein Me represents methyl group.

A metal-containing reagent represented by general formula (8) is preferable as a metal-containing reagent represented by the general formula (6), because the tertiary amine represented by the general formula (9) can be easily obtained using the above metal-containing reagent in accordance with reaction formula (13). When R⁶ is dialkoxymethyl group in general formula (8), R⁶ is formyl group in the tertiary amine represented by the general formula (9) obtained in accordance with reaction formula (13), and the dialkoxymethyl group includes diethoxymethyl group and dimethoxymethyl group.

R⁶—C≡C-M¹ (8)

wherein R⁶ represents alkyl group, aryl group, silyl group, vinyl group or dialkoxymethyl group having 2 or more carbon atoms, and M¹ represents an alkali metal atom.

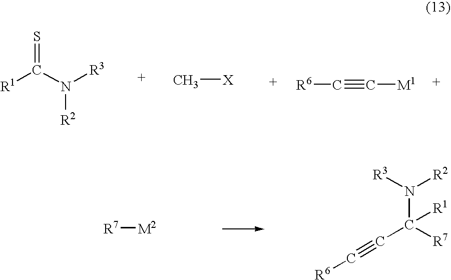

Any organic solvent may be used in the reaction with no problems, as long as it is commonly used in organic synthesis. Diethyl ether or tetrahydrofuran (THF) is preferable because these solvents do not inhibit the reaction of the components.

The reactions as shown in reaction formulas (10) and (13) progress at a reaction temperature of approximately 20° C. However, in order to improve production efficiency of the tertiary amine, that is, reaction efficiency in the reactions as shown in reaction formulas (10) and (13), when R¹ is hydrogen atom, the Grignard reagent is added preferably at a temperature (reaction temperature) between 0° C. and 35° C. On the other hand, when R¹ represents alkyl or aryl group, the above reagent is added preferably at a temperature between 40° C. and 70° C. If the reaction temperature is lower than the above preferred range, the progression of the reaction is slow, thereby reducing production efficiency. In contrast, if it is higher than the above preferred range, the solvent vaporizes.

Reaction time is also associated with such production efficiency of the tertiary amine. The reaction time is preferably between 15 minutes and 8 hours. If the reaction time is shorter than 15 minutes, the progression of the reaction is insufficient, thereby decreasing production efficiency. On the other hand, if the reaction time exceeds 8 hours, production efficiency decreases.

In one embodiment, there is provided a novel tertiary amine, especially an asymmetric tertiary amine, which can be used for pharmaceutical preparations, agricultural chemicals or chemical products.

In one embodiment, the tertiary amine represented by general formula (2) can be efficiently produced. The tertiary amine represented by general formula (2) has more types of physiological activities than those of the tertiary amine represented by general formula (1). Accordingly, it can be widely used for medical preparations or agricultural chemicals.

In one embodiment, the tertiary amine represented by general formula (3) can be produced without using catalysts. That is, the tertiary amine represented by general formula (3) can be produced only by adding in a solvent, thioamide, a methylating agent, a metal-containing reagent and a Grignard reagent in this order. It is not necessary to purify an intermediate product during the reaction. The tertiary amine can be produced at a temperature lower than ever before. Moreover, it is not necessary to pressurize the reaction system. Further, the yield of the tertiary amine is high. Accordingly, the method for producing tertiary amine in the present embodiment is easy.

In one embodiment, when the metal-containing reagent represented by general formula (8) is used, the tertiary amine represented by general formula (9) can be easily produced.

The above embodiment may be changed as follows:

The tertiary amine represented by general formula (1) may be used as a raw material for synthesis of various compounds. In this case, the tertiary amine acts as a source for amine ligands or the like, or it acts as a base compound synthesizing such ligands.

When the tertiary amine represented by general formula (3) is produced, a solvent containing thioamide and a methylating agent may be mixed with a solvent containing a metal-containing reagent, instead of adding the metal-containing reagent into the solvent containing the thioamide and the methylating agent.

Examples of the present invention will be described below.

### EXAMPLE 1

Diethyl ether (3 ml) was placed in a 20 ml two-necked flask that had been subjected to reduced-pressure drying and argon substitution. Thereafter, 0.13 ml (1.2 mmol) of phenylacetylene and 0.75 ml (1.2 mmol) of n-butyl lithium were added thereto at a temperature of 0° C., followed by stirring for 10 minutes, so as to obtain lithium acetylide. This reaction solution was referred as solution A.

Diethyl ether (3 ml) and N,N-dimethylthioformamide (0.085 ml (1.0 mmol)) were placed in a 50 ml two-necked flask that had been subjected to vacuum drying and argon substitution. Thereafter, 0.113 ml (1.0 mmol) of methyl trifluoromethanesulfonate was added thereto, followed by stirring at 20° C. for 30 seconds. This reaction solution was defined as solution B.

Solution A was added to solution B that had been cooled to 0° C. using an L-shaped tube. The mixed solution was stirred at 20° C. for 30 minutes, and 1.5 ml (1.5 mmol) of ethyl magnesium bromide was added thereto, followed by further stirring at 20° C. for 2 hours. Thereafter, ether extraction was carried out on the thus obtained reaction solution. The extract was washed with a saturated ammonium chloride aqueous solution, and then dried with anhydrous magnesium sulfate. Thereafter, filtration and concentration were carried out thereon, so as to obtain reddish-brown oil. The yield of the reddish-brown oil was 155 mg (83% yield), and the purity was 99% or higher. From the results of infrared absorption spectrometry, nuclear magnetic resonance spectrometry, and mass spectrometry, it was found that the reddish-brown oil of Example 1 was N,N-dimethyl-1-phenyl-1-pentyn-3-amine represented by structural formula (14).

<IR (KBr disk)> (neat)2936, 2872, 1489, 1041 cm⁻¹

<NMR (in CDCl₃, TMS internal standard)>

¹H-NMR: δ1.07(t, J=7.6 Hz, 3H, CH₃ in CH₂CH₃), 1.72(quint, J=7.5 Hz, 2H, CH₂ in CH₂CH₃), 2.34(s, 6H, NMe₂), 3.44(t, J=7.6 Hz, 1H, CH), 7.26-7.33(m, 3H, Ar), 7.43-7.45(m, 2H, Ar).

¹³C-NMR: δ11.3(CH₃), 27.1(CH₂), 41.8(NMe₂), 59.6(CH), 86.1, 86.8(C≡C), 123.4, 127.8, 128.2, 131.7(Ar).

<MS(EI)>

m/z=186(M⁺−1).

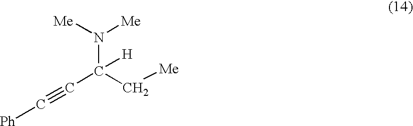

wherein Me represents methyl group, and Ph represents phenyl group.

### EXAMPLE 2

Solution A of Example 1 was added to solution B of Example 1 that had been cooled to 0° C., using an L-shaped tube, followed by stirring at 20° C. for 30 minutes. Subsequently, 1.5 ml (1.5 mmol) of phenyl magnesium bromide was added to the obtained reaction solution, followed by stirring at 20° C. for 2 hours. Dark red oil was obtained in the same manner as in Example 1. The yield of the dark red oil was 214 mg (91% yield), and the purity was 99% or higher. From the results of infrared absorption spectrometry, nuclear magnetic resonance spectrometry, and mass spectrometry, it was found that the dark red oil of Example 2 was N,N-dimethyl-α-(phenylethynyl)-benzenemethanamine represented by structural formula (15).

<IR (KBr disk)> (neat)2942, 2859, 2822, 1598, 1490, 1017 cm⁻¹

<NMR (in CDCl₃, TMS internal standard)>

¹H-NMR: δ2.33(s, 6H, NMe₂), 4.83(s, 1H, CH), 7.22-7.62(m, 10H, Ar).

¹³C-NMR: δ41.6(NMe₂), 62.2(CH), 84.7, 88.4(C≡C), 123.1, 127.2, 128.1, 128.2, 128.3, 128.4, 131.8, 138.6(Ar).

<MS(EI)>

m/z=235(M+).

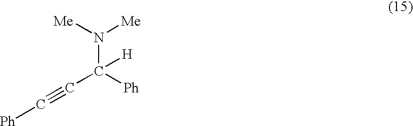

wherein Me represents methyl group, and Ph represents phenyl group.

### EXAMPLE 3

Dark red oil was obtained in the same manner as in Example 2 with the exception that 1.6 ml (1.5 mmol) of vinyl magnesium bromide was used instead of phenyl magnesium bromide. The yield of the dark red oil was 175 mg (95% yield), and the purity was 99% or higher. From the results of infrared absorption spectrometry, nuclear magnetic resonance spectrometry, and mass spectrometry, it was found that the dark red oil of Example 3 was N,N-dimethyl-5-phenyl-1-penten-4-yn-3-amine represented by structural formula (16).

<IR (KBr disk)> (neat)2940, 2859, 2780, 1490, 1031 cm⁻¹

<NMR (in CDCl₃, TMS internal standard)>

¹H-NMR: δ2.34(s, 6H, NMe₂), 4.25(dt, J=4.7, 1.7 Hz, 1H, CH), 5.31(dt, J=10.0, 1.7 Hz, 1H, CH₂ in CH═CH₂), 5.59(dt, J=17.2, 1.7 Hz, 1H, CH₂ in CH═CH₂), 5.93(ddd, J=17.1, 10.3, 4.7 Hz, 1H, CH in CH═CH₂), 7.29-7.34(m, 3H, Ar), 7.46-7.50(m, 2H, Ar).

¹³C-NMR: δ41.5(NMe₂), 60.5(CH), 88.3, 83.9(C≡C), 117.8(CH₂ in CH═CH₂), 123.1, 128.1, 128.3(Ar), 131.7(CH in CH═CH₂), 136.0(Ar).

<MS(EI)>

m/z=184 (M⁺−1).

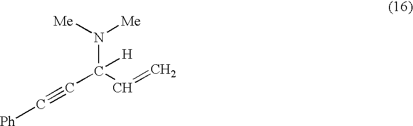

wherein Me represents methyl group, and Ph represents phenyl group.

### EXAMPLE 4

Solution A of Example 1 was added to solution B of Example 1 that had been cooled to 0° C., using an L-shaped tube, followed by stirring at 20° C. for 30 minutes. Subsequently, 3.0 ml (1.5 mmol) of ethynyl magnesium bromide was added to the obtained reaction solution, followed by stirring at 35° C. for 6 hours. Dark red oil was obtained in the same manner as in Example 1. The yield of the dark red oil was 167 mg (91% yield), and the purity was 99% or higher. From the results of infrared absorption spectrometry, nuclear magnetic resonance spectrometry, and mass spectrometry, it was found that the dark red oil of Example 4 was N,N-dimethyl-1-phenyl-1,4-pentadiyn-3-amine represented by structural formula (17).

<IR (KBr disk)> (neat)2947, 2861, 2784, 1490, 1039 cm⁻¹

<NMR (in CDCl₃, TMS internal standard)>

¹H-NMR: δ2.42(s, 6H, NMe₂), 2.44(d, J=2.2 Hz, 1H, CH in C≡CH), 4.57(d, J=2.2 Hz, 1H, CH), 7.26-7.33(m, 3H, Ar), 7.46-7.48(m, 2H, Ar).

¹³C-NMR: δ41.2(NMe₂), 49.4(CH), 72.6, 77.9, 83.2, 84.6(C≡C), 122.4, 128.3, 128.5, 131.9(Ar).

<MS(EI)>

m/z=182(M⁺−1).

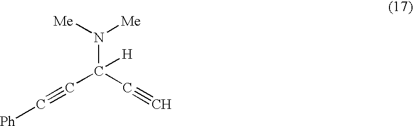

wherein Me represents methyl group, and Ph represents phenyl group.

### EXAMPLE 5

Solution A of Example 1 was added to solution B of Example 1 that had been cooled to 0° C., using an L-shaped tube, followed by stirring at 20° C. for 30 minutes. Subsequently, 1.7 ml (1.5 mmol) of butyl magnesium bromide was added to the obtained reaction solution, followed by stirring at 35° C. for 2 hours. Reddish-brown oil was obtained in the same manner as in Example 1. The yield of the reddish-brown oil was 212 mg (98% yield), and the purity was 99% or higher. From the results of infrared absorption spectrometry, nuclear magnetic resonance spectrometry, and mass spectrometry, it was found that the reddish-brown oil of Example 5 was N,N-dimethyl-1-phenyl-1-heptyn-3-amine represented by structural formula (18).

<IR (KBr disk)> (neat)2934, 2860, 2779, 1596, 1490, 1043 cm⁻¹

<NMR (in CDCl₃, TMS internal standard)>

¹H-NMR: δ0.93(t, J=6.8 Hz, 3H, CH₃ in CH₃(CH₂)₃), 1.33-1.56(m, 4H, (CH₂)₂ in CH₃(CH₂)₂CH₂), 1.71(quint, J=7.6 Hz, 2H, CH₂ in CH₃(CH₂)₂CH₂), 2.35(s, 6H, NMe₂), 3.54(t, J=7.6 Hz, 1H, CH), 7.26-7.33(m, 3H, Ar), 7.34-7.45(m, 2H, Ar).

¹³C-NMR: δ14.0 (CH₃ in CH₃(CH₂)₃), 22.5(CH₂ in CH₃CH₂(CH₂)₂), 28.9(CH₂ binding to C₂H₅ in C₂H₅CH₂CH₂), 33.7(CH₂ in CH₃(CH₂)₂CH₂), 41.4(NMe₂), 58.2(CH), 85.9, 87.1(C≡C), 123.4, 127.8, 128.2, 131.7(Ar).

<MS(EI)>

m/z=215 (M⁺)

<HRMS>

Calcd for C₁₅H₂₁N: 215.1674, Found: 215.1697.

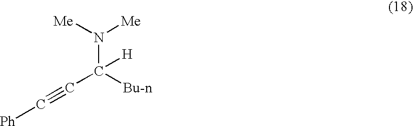

wherein Me represents methyl group, Ph represents phenyl group, and Bu-n represents n-butyl group.

### EXAMPLE 6

Reddish-brown oil was obtained in the same manner as in Example 2 with the exception that 0.75 ml (1.5 mmol) of isopropyl magnesium chloride was used instead of phenyl magnesium bromide. The yield of the reddish-brown oil was 178 mg (88%), and the purity was 99% or higher. From the results of infrared absorption spectrometry, nuclear magnetic resonance spectrometry, and mass spectrometry, it was found that the reddish-brown oil of Example 6 was N,N,4-trimethyl-1-phenyl-1-pentyn-3-amine represented by structural formula (19).

<IR (KBr disk)> (neat)2957, 1560, 1490, 1030 cm⁻¹

<NMR (in CDCl₃, TMS internal standard)>

¹H-NMR: δ1.03(d, J=6.3 Hz, 3H, (CH₃)₂ in CH(CH₃)₂), 1.12(d, J=6.4 Hz, 3H, (CH₃)₂ in CH(CH₃)₂), 1.86(heptd, J=9.8, 6.6 Hz, 1H, CH in CH(CH₃)₂), 2.30(s, 6H, NMe₂), 3.05(d, J=9.8 Hz, 1H, CH), 7.28-7.30 (m, 3H, Ar), 7.43-7.45(m, 2H, Ar).

¹³C-NMR: δ19.8, 20.6(CH₃), 31.0(CH in CH(CH₃)₂), 41.8(NMe₂), 65.6(CH), 85.6, 86.6(C≡C), 123.6, 127.8, 128.2, 131.7(Ar).

<MS(EI)>

m/z=200(M⁺−1).

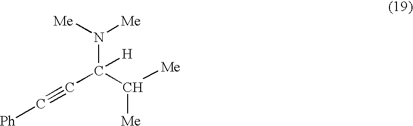

wherein Me represents methyl group, and Ph represents phenyl group.

### EXAMPLE 7

Reddish-brown oil was obtained in the same manner as in Example 2 with the exception that 1.5 ml (1.5 mmol) of allyl magnesium bromide was used instead of phenyl magnesium bromide. The yield of the reddish-brown oil was 164 mg (82% yield), and the purity was 99% or higher. From the results of infrared absorption spectrometry, nuclear magnetic resonance spectrometry, and mass spectrometry, it was found that the reddish-brown oil of Example 7 was N,N-dimethyl-6-phenyl-1-hexen-5-yn-4-amine represented by structural formula (20).

<IR (KBr disk)> (neat)2977, 2943, 2861, 2824, 1598, 1489, 1070 cm⁻¹

<NMR (in CDCl₃, TMS internal standard)>

¹H-NMR: δ2.34(s, 6H, NMe₂), 2.45-2.50(m, 2H, CH₂), 3.61(t, J=7.60 Hz, 1H, CH), 5.10-5.20(m, 2H, CH₂ in CH═CH₂), 5.93(ddd, J=17.2, 10.0, 7.2 Hz, 1H, CH in CH═CH₂), 7.28-7.34(m, 3H, Ar), 7.42-7.45(m, 2H, Ar).

¹³C-NMR: δ38.4(CH₂), 41.4(NMe₂), 58.0(CH), 86.2, 86.3(C≡C), 117.0(CH₂ in CH═CH₂), 123.2, 128.0, 128.2, 131.8(Ar), 135.0(CH in CH═CH₂).

<MS(EI)>

m/z=198 (M⁺−1).

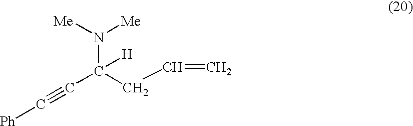

wherein Me represents methyl group, and Ph represents phenyl group.

### EXAMPLE 8

Diethyl ether (3 ml) was placed in a 20 ml two-necked flask that had been subjected to reduced-pressure drying and argon substitution. Thereafter, 0.12 ml (1.2 mmol) of trimethylsilylacetylene and 0.75 ml (1.2 mmol) of n-butyl lithium were added thereto at a temperature of 0° C., followed by stirring for 10 minutes, so as to obtain lithium acetylide. This reaction solution (solution C) was added to solution B of Example 1 that had been cooled to 0° C., using an L-shaped tube, followed by stirring at 20° C. for 30 minutes.

Subsequently, 1.5 ml (1.5 mmol) of phenyl magnesium bromide was added to the obtained reaction solution, followed by stirring at 20° C. for 2 hours. Then, reddish-brown oil was obtained in the same manner as in Example 1. The yield of the reddish-brown oil was 202 mg (87% yield), and the purity was 99% or higher. From the results of infrared absorption spectrometry, nuclear magnetic resonance spectrometry, and mass spectrometry, it was found that the reddish-brown oil of Example 8 was N,N-dimethyl-α-[(trimethylsilyl)ethynyl]-benzenemethanamine represented by structural formula (21).

<IR (KBr disk)> (neat)2958, 2859, 2780, 2162, 1492, 1021 cm⁻¹

<NMR (in CDCl₃, TMS internal standard)>

¹H-NMR: δ0.24(s, 9H, SiMe₃), 2.23(s, 6H, NMe₂), 4.60(s, 1H, CH), 7.16-7.35 (m, 3H, Ar), 7.52-760(m, 2H, Ar).

¹³C-NMR: δ0.23(SiMe₃), 41.4(NMe₂), 62.3(CH), 92.8, 100.8(C≡C), 127.6, 128.1, 128.4, 138.3(Ar).

<MS(EI)>

m/z=231 (M⁺).

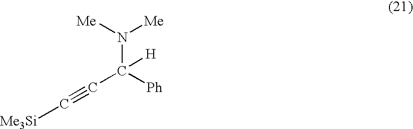

wherein Me represents methyl group, and Ph represents phenyl group.

### EXAMPLE 9

Solution C of Example 8 was added to solution B of Example 1 that had been cooled to 0° C., using an L-shaped tube, followed by stirring at 20° C. for 30 minutes. Subsequently, 1.5 ml (1.5 mmol) of allyl magnesium bromide was added to the obtained reaction solution, followed by stirring at 20° C. for 2 hours. Red oil was obtained in the same manner as in Example 1. The yield of the red oil was 149 mg (76% yield), and the purity was 99% or higher. From the results of infrared absorption spectrometry, nuclear magnetic resonance spectrometry, and mass spectrometry, it was found that the red oil of Example 9 was N,N-dimethyl-6-(trimethylsilyl)-1-hexen-5-yn-4-amine represented by structural formula (22).

<IR (KBr disk)> (neat)2960, 2825, 2781, 2160, 1457, 1024 cm⁻¹

<NMR (in CDCl₃, TMS internal standard)>

¹H-NMR: δ0.17(s, 9H, SiMe₃), 2.24(s, 6H, NMe₂), 2.36(td, J=7.6, 1.2 Hz, 2H, CH₂), 3.37(t, J=7.6 Hz, 1H, CH), 5.06-5.14(m, 2H, CH₂ in CH═CH₂), 5.86(ddd, J=17.4, 10.0, 7.6 Hz, 1H, CH in CH═CH₂).

¹³C-NMR: δ0.22(SiMe₃), 38.3(CH₂), 41.2(NMe₂), 58.1(CH), 90.3, 102.7(C≡C), 116.8(CH₂ in CH═CH₂), 135.0(CH in CH═CH₂).

<MS (EI)>

m/z=195(M⁺−1).

<HRMS>

Calcd for C₁₁H₂₁NSi: 195.14433, Found: 195.14578.

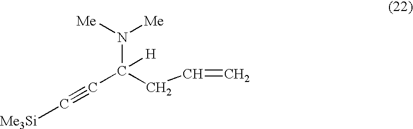

wherein Me represents methyl group.

### EXAMPLE 10

Diethyl ether (3 ml) was placed in a 20 ml two-necked flask that had been subjected to reduced-pressure drying and argon substitution. Thereafter, 0.14 ml (1.2 mmol) of 1-hexyne and 0.75 ml (1.2 mmol) of n-butyl lithium were added thereto at a temperature of 0° C., followed by stirring for 10 minutes, so as to obtain lithium acetylide. This reaction solution (solution D) was added to solution B of Example 1 that had been cooled to 0° C., using an L-shaped tube, followed by stirring at 20° C. for 30 minutes.

Subsequently, 1.5 ml (1.5 mmol) of phenyl magnesium bromide was added to the obtained reaction solution, followed by stirring at 20° C. for 2 hours. Then, reddish-brown oil was obtained in the same manner as in Example 1. The yield of the reddish-brown oil was 194 mg (90% yield), and the purity was 99% or higher. From the results of infrared absorption spectrometry, nuclear magnetic resonance spectrometry, and mass spectrometry, it was found that the reddish-brown oil of Example 10 was N,N-dimethyl-α-(1-hexynyl)-benzenemethanamine represented by structural formula (23).

<IR (KBr disk)> (neat)2957, 2934, 2860, 2778, 2256, 1492, 1044 cm⁻¹

<NMR (in CDCl₃, TMS internal standard)>

¹H-NMR: δ0.94(t, J=7.2 Hz, 3H, CH₃ in CH₃(CH₂)₃), 1.50(sext, J=7.7 Hz, 2H, CH₂ binding to (CH₂)₂ in CH₃CH₂(CH₂)₂), 1.56(quint, J=6.9 Hz, 2H, CH₂ binding to C₂H₅ in C₂H₅CH₂CH₂), 2.23(s, 6H, NMe₂), 2.33(t, J=6.8 Hz, 2H, CH₂ binding to (CH₂)₂ in CH₃(CH₂)₂CH₂), 4.57(s, 1H, CH), 7.26-7.52(m, 3H, Ar), 7.54-7.55(m, 2H, Ar).

¹³C-NMR: 613.6(CH₃ in CH₃(CH₂)₃), 18.5(CH₂ binding to (CH₂)₂ in CH₃CH₂ (CH₂)₂), 22.0 (CH₂ binding to C₂H₅ in C₂H₅CH₂CH₂), 31.2(CH₂ binding to (CH₂)₂ in CH₃(CH₂)₂CH₂), 41.5(NMe₂), 61.8(CH), 74.8, 88.6(C≡C), 127.5, 128.1, 128.5, 139.3(Ar).

<MS(EI)>

m/z=215 (M⁺).

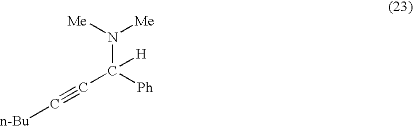

wherein Me represents methyl group, Ph represents phenyl group, and n-Bu represents n-butyl group.

### EXAMPLE 11

Diethyl ether (3 ml) was placed in a 20 ml two-necked flask that had been subjected to reduced-pressure drying and argon substitution. Thereafter, 0.14 ml (1.2 mmol) of 2-methyl-1-buten-3-yne and 0.75 ml (1.2 mmol) of n-butyl lithium were added thereto at a temperature of 0° C. , followed by stirring for 10 minutes, so as to obtain lithium acetylide. This reaction solution (solution E) was added to solution B of Example 1 that had been cooled to 0° C. , using an L-shaped tube, followed by stirring at 20° C. for 30 minutes.

Subsequently, 1.5 ml (1.5 mmol) of phenyl magnesium bromide was added to the obtained reaction solution, followed by stirring at 20° C. for 2 hours. Then, reddish-brown oil was obtained in the same manner as in Example 1. The yield of the reddish-brown oil was 173 mg (87% yield), and the purity was 99% or higher. From the results of infrared absorption spectrometry, nuclear magnetic resonance spectrometry, and mass spectrometry, it was found that the reddish-brown oil of Example 11 was N,N-dimethyl-α-(2-methyl-1-buten-3-ynyl)-benzenemethanamine represented by structural formula (24).

<IR (KBr disk)> (neat)2945, 2859, 2822, 2779, 1491, 1043 cm⁻¹

<NMR (in CDCl₃, TMS internal standard)>

¹H-NMR: δ1.97(s, 3H, CH₃), 2.26(s, 6H, NMe₂), 4.71(s, 1H, CH), 5.26(quint, J=1.7 Hz, 1H, CH₂ in C═CH₂), 5.36(s, 1H, CH₂ in C═CH₂), 7.28-7.37(m, 3H, Ar), 7.53-7.55(m, 2H, Ar).

¹³C-NMR: δ23.9(CH₃), 41.5(NMe₂), 62.1(CH), 83.7, 89.6(C≡C), 121.6, 126.7(C═C), 127.7, 128.2, 128.4, 138.7(Ph).

<MS(EI)>

m/z=198 (M⁺−1).

<HRMS>

Calcd for C₁₄H₁₇N: 199.1361, Found: 199.1350.

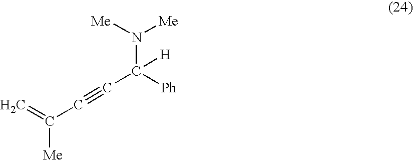

wherein Me represents methyl group, and Ph represents phenyl group.

### EXAMPLE 12

Diethyl ether (3 ml) was placed in a 20 ml two-necked flask that had been subjected to reduced-pressure drying and argon substitution. Thereafter, 0.17 ml (1.2 mmol) of propargyl aldehyde diethyl acetal and 0.75 ml (1.2 mmol) of n-butyl lithium were added thereto at a temperature of 0° C., followed by stirring for 10 minutes, so as to obtain lithium acetylide. This reaction solution (solution F) was added to solution B of Example 1 that had been cooled to 0° C., using an L-shaped tube, followed by stirring at 20° C. for 30 minutes.

Subsequently, 1.5 ml (1.5 mmol) of phenyl magnesium bromide was added to the obtained reaction solution, followed by stirring at 20° C. for 2 hours. Then, dark red oil was obtained in the same manner as in Example 1. The yield of the dark red oil was 250 mg (96% yield), and the purity was 99% or higher. From the results of infrared absorption spectrometry, nuclear magnetic resonance spectrometry, and mass spectrometry, it was found that the dark red oil of Example 12 was N,N-dimethyl-α-(3,3-diethoxy-1-propynyl)-benzenemethanamine represented by structural formula (25).

<IR (KBr disk)> (neat)2976, 2824, 2780, 1450, 1052 cm⁻¹

<NMR (in CDCl₃, TMS internal standard)>

¹H-NMR: δ1.26(td, J=7.0, 1.0 Hz, 6H, CH₃ in CH₂CH₃), 2.26(s, 6H, NMe₂), 3.65(m, 2H, CH₂ in CH₂CH₃), 3.81(m, 2H, CH₂ in CH₂CH₃), 4.69(s, 1H, CH in CH(OCH₂CH₃)₂), 5.43(d, J=1.6 Hz, 1H, CH), 7.28-7.36(m, 3H, Ar), 7.52-7.54(m, 2H, Ar).

¹³C-NMR: δ15.2(CH₃ in CH₂CH₃), 41.6(NMe₂), 60.9(CH), 61.7(CH₂ in CH(OCH₂CH₃)₂), 80.6, 83.9(C≡C), 91.5(CH in CH(OCH₂CH₃)₂), 127.2, 128.2, 128.3, 138.1(Ar).

<MS(EI)>

m/z=216(M⁺−1).

<HRMS>

Calcd for C₁₆H₂₃NO₂: 261.17288, Found: 261.17453.

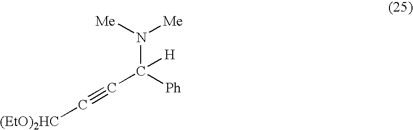

wherein Me represents methyl group, Ph represents phenyl group, and Et represents ethyl group.

### EXAMPLE 13

Diethyl ether (3 ml) and 0.141 g (1.0 mmol) of N,N-diallylthioformamide were placed in a 50 ml two-necked flask that had been subjected to reduced-pressure drying and argon substitution. Thereafter, 0.113 ml (1.0 mmol) of methyl trifluoromethanesulfonate was added thereto, followed by stirring at 20° C. for 30 seconds. This reaction solution was defined as solution G.

Using an L-shaped tube, solution A of Example 1 was added to solution G that had been cooled to 0° C., followed by stirring at 20° C. for 30 minutes. Subsequently, 1.5 ml (1.5 mmol) of phenyl magnesium bromide was added to the obtained reaction solution, followed by stirring at 20° C. for 2 hours. Thereafter, ether extraction was carried out on the thus obtained reaction solution. The extract was washed with a saturated ammonium chloride aqueous solution, and then dried with anhydrous magnesium sulfate. Thereafter, drying and concentration were carried out thereon, and the obtained product was then subjected to silica gel column chromatography for purification (as a developing solvent, hexane:ethyl acetate=20:1 (volume ratio), Rf=0.46), so as to obtain yellow oil. The yield of the yellow oil was 195 mg (68% yield). From the results of infrared absorption spectrometry, nuclear magnetic resonance spectrometry, and mass spectrometry, it was found that the yellow oil of Example 13 was N,N-(di-2-propenyl)-α-(phenylethynyl)-benzenemethanamine represented by structural formula (26).

<IR (KBr disk)> (neat)3079, 3031, 2978, 2924, 2817, 1490, 1029 cm⁻¹

<NMR (in CDCl₃, TMS internal standard)>

¹H-NMR: δ3.05(dd, J=14.2, 7.7 Hz, 2H, CH₂), 3.28(ddt, J=14.2, 4.4, 1.5 Hz, 2H, CH₂), 5.10(s, 1H, CH), 5.13(d, J=17.5 Hz, 2H, CH₂ in CH═CH₂), 5.27(dd, J=17.5, 1.5 Hz, 2H, CH₂ in CH═CH₂), 5.85(dddd, J=20.0, 10.4, 7.7, 4.4 Hz, 2H, CH in CH═CH₂), 7.27-7.37(m, 6H, Ar), 7.52-7.56(m, 2H, Ar), 7.68(d, J=7.2 Hz, 2H, Ar).

¹³C-NMR: 653.6(CH₂), 56.6(CH), 87.4, 87.9(C≡C), 117.3(CH₂ in CH═CH₂), 127.4, 128.1, 128.2, 128.3, 128.4, 128.5, 131.9(Ar), 136.5(CH in CH═CH₂), 139.4(Ar).

<MS(EI)>

m/z=286(M⁺−1).

<HRMS>

Calcd for C₂₁H₂₁N: 287.16740, Found: 287.16511.

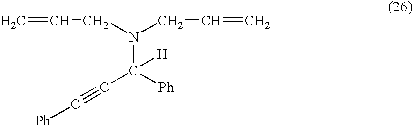

wherein Ph represents phenyl group.

### EXAMPLE 14

Diethyl ether (8 ml), N,N-dimethylthioformamide (0.085 ml (1 mmol)), and methyl trifluoromethanesulfonate (0.115 ml (1 mmol)) were successively placed in a 20 ml two-necked flask that had been subjected to reduced-pressure drying and argon substitution, followed by stirring at 20° C. for 30 seconds. Thereafter, this reaction solution was cooled to 0° C. , and 1.6 ml (0.94 M solution in Et₂O; 1.5 mmol) of phenyl lithium was added thereto, followed by stirring at 20° C. for 1 hour. This reaction solution was defined as solution H. 2.0 ml (1.0 M solution in THF; 2 mmol) of ethyl magnesium bromide was added to solution H, followed by stirring at 20° C. for 3 hours. 20 ml of saturated ammonium chloride aqueous solution was further added thereto, and the reaction was then terminated. Thereafter, ether extraction was repeatedly carried out 3 times on the thus obtained reaction solution, and extraction of the ether layer was repeatedly carried out 3 times using 6 ml of concentrated hydrochloric acid. Subsequently, the obtained extract was adjusted to alkaline pH (pH=13 to 14) with a 30% sodium hydroxide aqueous solution, and ether extraction was repeatedly carried out 5 times thereon using 6 ml of diethyl ether. Thereafter, the extract was dried with anhydrous magnesium sulfate, followed by filtration and concentration, so as to obtain light yellow oil. The yield of the light yellow oil was 0.095 g (58% yield). From the results of nuclear magnetic resonance spectrometry, it was found that the light yellow oil of Example 14 was N,N-dimethyl-α-ethyl-benzenemethanamine represented by structural formula (27).

<NMR (in CDCl₃, TMS internal standard)>

¹H-NMR: δ0.72(t, J=7.6 Hz, 3H, CH₃), 1.71-1.82(m, 1H, CH₂), 1.91-2.03(m, 1H, CH₂), 2.20(s, 6H, CH₃), 3.09(dd, J=4.7, 9.8 Hz, 1H, CH), 7.20-7.28(m, 3H, Ar), 7.30-7.37(m, 2H, Ar).

¹³C-NMR: δ11.0(CH₃), 26.0(CH₂), 42.9(N(CH₃)₂), 72.7(CH), 127.1, 128.1, 128.7, 140.1(Ar).

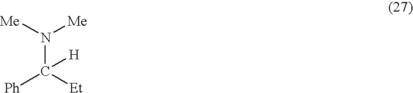

wherein Me represents methyl group, Ph represents phenyl group, and Et represents ethyl group.

### EXAMPLE 15

A light yellow solid was obtained in the same manner as in Example 14 with the exception that 2.0 ml (1.0 M solution in THF; 2 mmol) of phenyl magnesium bromide was used instead of ethyl magnesium bromide. The yield of the light yellow solid was 0.186 g (88% yield). From the results of nuclear magnetic resonance spectrometry, it was found that the light yellow solid of Example 15 was N,N-dimethyl-α-phenyl-benzenemethanamine represented by structural formula (28).

<NMR (in CDCl₃, TMS internal standard)>

¹H-NMR: δ2.18(s, 6H, N(CH₃)₂), 4.05(s, 1H, CH), 7.15(t, J=7.2 Hz, 2H, Ar), 7.25(t, J=7.2 Hz, 4H, Ar), 7.42(t, J=7.2 Hz, 4H, Ar).

¹³C-NMR: δ44.7(N(CH₃)₂), 78.0(CH), 126.9, 127.7, 128.4, 143.4(Ar).

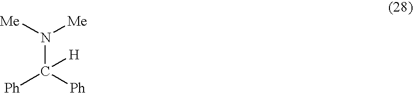

wherein Me represents methyl group, and Ph represents phenyl group.

### EXAMPLE 16

Light brown oil was obtained in the same manner as in Example 14 with the exception that 2.0 ml (1.0 M solution in Et₂O; 2 mmol) of allyl magnesium bromide was used instead of ethyl magnesium bromide. The yield of the light brown oil was 0.130 g (74% yield). From the results of nuclear magnetic resonance spectrometry, it was found that the light brown oil of Example 16 was N,N-dimethyl-α-2-propenyl-benzenemethanamine represented by structural formula (29).

<NMR (in CDCl₃, TMS internal standard)>

H-NMR: δ2.19(s, 6H, N(CH₃)₂), 2.48-2.57(m, 1H, CH₂ having a single bond with CH of CH₂═CHCH₂), 2.61-2.69(m, 1H, CH₂ having a single bond with CH of CH₂═CHCH₂), 4.92(dt, J=1.2, 10.0 Hz, 1H, CH₂ having a double bond with CH of CH₂═CHCH₂), 4.98(dq, J=2.0, 17.2 Hz, 1H, CH₂ having a double bond with CH of CH₂═CHCH₂), 5.61(ddt, J=6.8, 10.4, 17.2 Hz, 1H, CH in CH₂═CHCH₂).

¹³C-NMR: δ37.8(CH₂ having a single bond with CH of CH₂═CHCH₂), 42.7(N(CH₃)₂), 70.6(CH), 116.4(CH₂ having a double bond with CH of CH₂═CHCH₂), 127.7, 128.0, 128.6(Ar), 135.7(CH in CH₂═CHCH₂), 139.0(Ar)

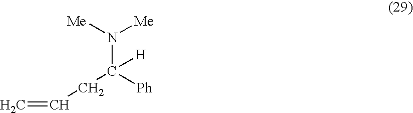

wherein Me represents methyl group, and Ph represents phenyl group.

### EXAMPLE 17

Dark brown oil was obtained in the same manner as in Example 14 with the exceptions that 4.0 ml (0.5 M solution in THF; 2 mmol) of ethynyl magnesium bromide was used instead of ethyl magnesium bromide, and that ethynyl magnesium bromide was added to solution H followed by stirring at 70° C. for 3 hours. The yield of the dark brown oil was 0.186 g (88% yield). From the results of nuclear magnetic resonance spectrometry, it was found that the dark brown oil of Example 17 was N,N-dimethyl-α-ethynyl-benzenemethanamine represented by structural formula (30).

<NMR (in CDCl₃, TMS internal standard)>

¹H-NMR: δ2.23(s, 6H, CH₃), 2.62(d, J=2.4 Hz, 1H, CH in C≡CH), 4.64(d, J=2.4 Hz, 1H, PhCH), 7.26-7.39(m, 3H, Ar), 7.56-7.58(m, 2H, Ar).

¹³C-NMR: δ41.5(N(CH₃)₂), 61.8(CH), 76.1(CH in C≡CH), 79.2(C in C≡CH), 128.0, 128.4, 128.6, 138.9(Ar).

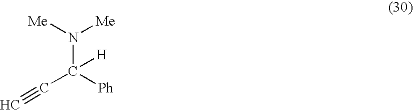

wherein Me represents methyl group, and Ph represents phenyl group.

### EXAMPLE 18

Diethyl ether (8 ml), N,N-dimethyl-4-bromobenzenecarbothioamide (0.244 g (1 mmol)), and methyl trifluoromethanesulfonate (0.115 ml (1 mmol)) were successively placed in a 20 ml two-necked flask that had been subjected to reduced-pressure drying and argon substitution, followed by stirring at 20° C. for 30 seconds. Then, this reaction solution was cooled to 0° C., and 1.6 ml (0.94 M solution in Et₂O; 1.5 mmol) of phenyl lithium was added thereto, followed by stirring at 20° C. for 1 hour. Thereafter, 2.0 ml (1.0 M solution in THF; 2 mmol) of ethyl magnesium bromide was added to the reaction solution, followed by stirring at 20° C. for 3 hours. Thereafter, 20 ml of saturated ammonium chloride aqueous solution was further added thereto, and the reaction was then terminated. Thereafter, ether extraction was repeatedly carried out 3 times on the thus obtained reaction solution, and extraction of the ether layer was repeatedly carried out 3 times thereon using 6 ml of concentrated hydrochloric acid. Subsequently, the obtained extract was adjusted to alkaline pH (pH=13 to 14) with a 30% sodium hydroxide aqueous solution, and ether extraction was repeatedly carried out 5 times thereon using 6 ml of diethyl ether. Thereafter, the extract was dried with anhydrous magnesium sulfate, followed by filtration and concentration, so as to obtain a yellow solid. The yield of the yellow solid was 0.167 g (52% yield). From the results of infrared absorption spectrometry and nuclear magnetic resonance spectrometry, it was found that the yellow solid of Example 18 was N,N-dimethyl-α-(4-bromophenyl)-α-ethyl-benzenemethanamine represented by structural formula (31).

<IR (KBr disk)> 3085, 3057, 3022, 2981, 2936, 2863, 2824, 2782, 1664, 1586, 1484, 1446, 1394, 1009, 823, 758, 706 cm⁻¹

<NMR (in CDCl₃, TMS internal standard)>

¹H-NMR: δ0.59(t, J=7.2 Hz, CH₃), 2.19(s, 6H, N(CH₃)₂), 2.06-2.21(m, 2H, CH₂), 7.21(d, J=8.8 Hz, 2H, Ar), 7.25-7.34(m, 5H, Ar), 7.43(d, J=8.8 Hz, 2H, Ar).

¹³C-NMR: δ8.5(CH₃), 31.7(CH₂), 39.4(N(CH₃)₂), 120.0(C), 126.5, 127.1, 129.5, 130.1, 131.4, 139.7, 140.2(Ar).

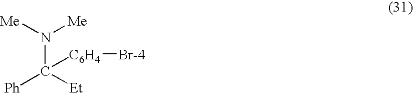

wherein Me represents methyl group, Ph represents phenyl group, and C₆H₄-Br-4 represents 4-bromophenyl group.

### EXAMPLE 19

Diethyl ether (8 ml), N,N-dimethylthiobenzamide (0.165 g (1 mmol)), and methyl trifluoromethanesulfonate (0.115 ml (1 mmol)) were successively placed in a 20 ml two-necked flask that had been subjected to reduced-pressure drying and argon substitution, followed by stirring at 20° C. for 30 seconds. Thereafter, this reaction solution was cooled to 0° C. , and 0.94 ml (1.6 M solution in hexane; 1.5 mmol) of butyl lithium was added thereto, followed by stirring at 20° C. for 1 hour. 2.0 ml (1.0 M solution in Et₂O; 2 mmol) of allyl magnesium bromide was further added to the reaction solution, followed by stirring at 20° C. for 3 hours. Thereafter, 20 ml of saturated ammonium chloride aqueous solution was further added thereto, and the reaction was then terminated. Thereafter, ether extraction was repeatedly carried out 3 times on the thus obtained reaction solution, and extraction of the ether layer was repeatedly carried out 3 times thereon using 6 ml of concentrated hydrochloric acid. Subsequently, the obtained extract was adjusted to alkaline pH (pH=13 to 14) with a 30% sodium hydroxide aqueous solution, and ether extraction was repeatedly carried out 5 times thereon using 6 ml of diethyl ether. Thereafter, the extract was dried with anhydrous magnesium sulfate, followed by filtration and concentration, so as to obtain yellow oil. The yield of the yellow oil was 0.136 g (59% yield). From the results of infrared absorption spectrometry and nuclear magnetic resonance spectrometry, it was found that the yellow oil of Example 19 was N,N-dimethyl-α-butyl-α-2-propenyl-benzenemethanamine represented by structural formula (32).

<IR (KBr disk)> (neat)3060, 2954, 2870, 2823, 2780, 1688, 1637, 1598, 1445, 911, 765 cm⁻¹

<NMR (in CDCl₃, TMS internal standard)>

¹H-NMR: δ0.86(t, J=7.2 Hz, 3H, CH₃ in CH₂CH₂CH₂CH₃), 1.07-1.31(m, 4H, (CH₂)₂ in CH₂(CH₂)₂CH₃), 1.83-1.88(m, 2H, CH₂ binding to (CH₂)₂ in CH₂(CH₂)₂CH₃), 2.19(s, 6H, N(CH₃)₂), 2.66-2.79(m, 2H, CH₂ having a single bond with CH of CH₂═CHCH₂), 5.02(dq, J=1.2, 10.0 Hz, 1H, CH₂ having a double bond with CH of CH₂═CHCH₂), 5.10(dq, J=1.6, 17.2 Hz, 1H, CH₂ having a double bond with CH of CH₂═CHCH₂), 5.83(ddt, J=7.6, 10.0, 17.2 Hz, 1H, CH in CH₂═CHCH₂), 7.20-7.26(m, H, Ar), 7.30-7.34(m, 2H, Ar), 7.38-7.40(m, 2H, Ar).

¹³C-NMR: δ14.1(CH₃ in CH₂CH₂CH₂CH₃), 23.5, 26.3, 35.4, 38.4(CH₂), 39.1(N(CH₃)₂), 63.9(C), 116.6(CH₂ having a double bond with CH of CH₂═CHCH₂), 126.1, 127.5, 127.6(Ar), 135.8(CH in CH₂═CHCH₂), 142.1(Ar).

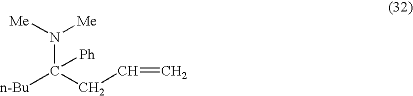

wherein Me represents methyl group, Ph represents phenyl group, and n-Bu represents n-butyl group.

### EXAMPLE 20

Diethyl ether (5 ml) was placed in a 20 ml two-necked flask that had been subjected to reduced-pressure drying and argon substitution. Thereafter, 0.21 ml (1.5 mmol) of trimethylsilylacetylene and 0.94 ml (1.6 M solution in hexane; 1.5 mmol) of butyl lithium were added thereto at a temperature of 0° C., followed by stirring for 10 minutes, so as to obtain lithium acetylide. This reaction solution was defined as solution I. At the same time, 5 ml of diethyl ether, 0.103 g (1 mmol) of N,N-dimethylthioacetoamide, and 0.115 ml (1 mmol) of methyl trifluoromethanesulfonate were successively placed in a 50 ml two-necked flask that had been subjected to reduced-pressure drying and argon substitution, followed by stirring at 20° C. for 30 seconds. This reaction solution was defined as solution J.

Solution I was added to solution J that had been cooled to 0° C., using an L-shaped tube, followed by stirring at 20° C. for 30 minutes. Subsequently, 10 ml (1.0 M solution in Et₂O; 10 mmol) of phenyl magnesium bromide was added to the obtained reaction solution, followed by stirring at 70° C. for 6 hours. Thereafter, 20 ml of saturated ammonium chloride aqueous solution was further added thereto, and the reaction was then terminated. Thereafter, ether extraction was repeatedly carried out 3 times on the thus obtained reaction solution, and extraction of the ether layer was repeatedly carried out 3 times thereon using 6 ml of concentrated hydrochloric acid.

Subsequently, the obtained extract was adjusted to alkaline pH (pH=13 to 14) with a 30% sodium hydroxide aqueous solution, and ether extraction was repeatedly carried out 5 times thereon using 6 ml of diethyl ether. Further, extraction of the ether layer was repeatedly carried out 3 times thereon using 6 ml of concentrated hydrochloric acid. The obtained extract was adjusted to alkaline pH (pH=13 to 14) with a 30% sodium hydroxide aqueous solution, and ether extraction was repeatedly carried out 5 times thereon using 6 ml of diethyl ether. Thereafter, the extract was dried with anhydrous magnesium sulfate, followed by filtration and concentration, so as to obtain yellow oil. The yield of the yellow oil was 0.167 g (68% yield). From the results of infrared absorption spectrometry and nuclear magnetic resonance spectrometry, it was found that the yellow oil of Example 20 was N,N-dimethyl-α-methyl-α-[(trimethylsilyl)ethynyl]-benzenemethanamine represented by structural formula (33).

<IR (KBr disk)> (neat)3060, 3026, 2986, 2956, 2862, 2823, 2782, 2158, 1600, 1489, 1447, 1250, 928, 843, 762, 700 cm⁻¹

<NMR (in CDCl₃, TMS internal standard)>

¹H-NMR: δ0.25(s, 9H, SiMe₃), 1.56(s, 3H, CH₃), 2.17(s, 6H, N(CH₃)₂), 7.21-7.25(m, 1H, Ar), 7.26-7.34(m, 2H, Ar), 7.66-7.69(m, 2H, Ar).

¹³C-NMR: δ0.36(SiMe₃), 31.2(CH₃), 40.3(N(CH₃)₂), 64.0(C), 91.7, 104.1(C≡C), 126.3, 127.0, 128.0, 145.0(Ar).

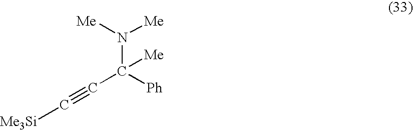

wherein Me represents methyl group, and Ph represents phenyl group.

### EXAMPLE 21

Diethyl ether (5 ml), N,N,2-trimethylpropanethioamide (0.131 g (1 mmol)), and 0.115 ml (1 mmol) of methyl trifluoromethanesulfonate were successively placed in a 50 ml two-necked flask that had been subjected to reduced-pressure drying and argon substitution, followed by stirring at 20° C. for 30 seconds. This reaction solution was defined as solution K.

Using an L-shaped tube, solution I of Example 20 was added to solution K that had been cooled to 0° C., followed by stirring at 20° C. for 30 minutes. Subsequently, 10 ml (1.0 M solution in Et₂O; 10 mmol) of allyl magnesium bromide was added to the obtained reaction solution, followed by stirring at 20° C. for 6 hours. Thereafter, 20 ml of saturated ammonium chloride aqueous solution was further added thereto, and the reaction was then terminated. Thereafter, ether extraction was repeatedly carried out 3 times on the thus obtained reaction solution, and extraction of the ether layer was repeatedly carried out 3 times thereon using 6 ml of concentrated hydrochloric acid. Subsequently, the obtained extract was adjusted to alkaline pH (pH=13 to 14) with a 30% sodium hydroxide aqueous solution, and ether extraction was repeatedly carried out 5 times thereon using 6 ml of diethyl ether. Further, extraction of the ether layer was repeatedly carried out 3 times thereon using 6 ml of concentrated hydrochloric acid. The obtained extract was adjusted to alkaline pH (pH=13 to 14) with a 30% sodium hydroxide aqueous solution, and ether extraction was repeatedly carried out 5 times thereon using 6 ml of diethyl ether. Thereafter, the extract was dried with anhydrous magnesium sulfate, followed by filtration and concentration, so as to obtain yellow oil. The yield of the yellow oil was 0.100 g (44% yield). From the results of infrared absorption spectrometry and nuclear magnetic resonance spectrometry, it was found that the yellow oil of Example 21 was N,N-dimethyl-4-(1-methylethyl)-6-(trimethylsilyl)-1-hexen-5-yn-4-amine represented by structural formula (34).

<IR (KBr disk)> (neat)3076, 2961, 2825, 2785, 2155, 1637, 1536, 1468, 1250, 857, 842 cm⁻¹

<NMR (in CDCl₃, TMS internal standard)>

¹H-NMR: δ0.17(s, 9H, SiMe₃), 0.97(d, J=6.8 Hz, 3H, (CH₃)₂ in CH(CH₃)₂), 1.06(d, J=6.8 Hz, 3H, (CH₃)₂ in CH(CH₃)₂), 2.05(sept, J=6.8 Hz, 1H, CH in CH(CH₃)₂), 2.30(s, 6H, N(CH₃)₂), 2.38-2.46(m, 2H, CH₂ having a single bond with CH of CH₂═CHCH₂), 4.99(dq, J=1.2, 10.4 Hz, 1H, CH₂ having a double bond with CH of CH₂═CHCH₂), 5.04(dq, J=2.0, 17.2 Hz, 1H, CH₂ having a double bond with CH of CH₂═CHCH₂), 6.02(ddt, J=6.8, 10.0, 17.2 Hz, 1H, CH in CH₂═CHCH₂)

¹³C-NMR: δ0.33(SiMe₃), 17.2, 19.1((CH₃)₂ in CH(CH₃)₂), 34.1(CH in CH(CH₃)₂), 37.5(CH₂ having a single bond with CH of CH₂═CHCH₂), 40.0(N(CH₃)₂), 65.4(C), 89.8, 106.3(C≡C), 115.6(CH₂ having a double bond with CH of CH₂═CHCH₂), 136.7(CH in CH₂═CHCH₂).

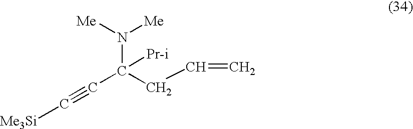

wherein Me represents methyl group, and Pr-i represents isopropyl group.

### EXAMPLE 22

Diethyl ether (5 ml), N,N,-dimethylthiobenzamide (0.165 g (1 mmol)), and methyl trifluoromethanesulfonate (0.115 ml (1 mmol)) were successively placed in a 50 ml two-necked flask that had been subjected to reduced-pressure drying and argon substitution, followed by stirring at 20° C. for 30 seconds. This reaction solution was defined as solution L.

Using an L-shaped tube, solution I of Example 20 was added to solution L that had been cooled to 0° C., followed by stirring at 20° C. for 30 minutes. Subsequently, 10 ml (1.0 M solution in THF; 10 mmol) of ethyl magnesium bromide was added to the obtained reaction solution, followed by stirring at 70° C. for 6 hours. Thereafter, 20 ml of saturated ammonium chloride aqueous solution was further added thereto, and the reaction was then terminated. Thereafter, ether extraction was repeatedly carried out 3 times on the thus obtained reaction solution, and extraction of the ether layer was repeatedly carried out 3 times thereon using 6 ml of concentrated hydrochloric acid. Subsequently, the obtained extract was adjusted to alkaline pH (pH=13 to 14) with a 30% sodium hydroxide aqueous solution, and ether extraction was repeatedly carried out 5 times thereon using 6 ml of diethyl ether. Further, extraction of the ether layer was repeatedly carried out 3 times thereon using 6 ml of concentrated hydrochloric acid. The obtained extract was adjusted to alkaline pH (pH=13 to 14) with a 30% sodium hydroxide aqueous solution, and ether extraction was repeatedly carried out 5 times thereon using 6 ml of diethyl ether. Thereafter, the extract was dried with anhydrous magnesium sulfate, followed by filtration and concentration, so as to obtain yellow oil. The yield of the yellow oil was 0.192 g (73% yield). From the results of infrared absorption spectrometry and nuclear magnetic resonance spectrometry, it was found that the yellow oil of Example 22 was N,N-dimethyl-α-ethyl-α-[(trimethylsilyl)ethynyl]-benzenemethanamine represented by structural formula (35).

<IR (KBr disk)> (neat)3061, 3025, 2958, 2864, 2824, 2783, 2155, 1600, 1448, 1250, 858, 842, 760, 700 cm⁻¹

<NMR (in CDCl₃, TMS internal standard)>

¹H-NMR: δ0.25(s, 9H, SiMe₃), 0.62(t, J=7.4 Hz, 3H, CH₃), 1.77(dq, J=7.4, 12.8 Hz, 1H, CH₂), 2.05(dq, J=7.4, 12.8 Hz, 1H, CH₂), 2.18(s, 6H, N(CH₃)₂), 7.21-7.25(m, 1H, Ar), 7.29-7.33(m, 2H, Ar), 7.59-7.62(m, 2H, Ar).

¹³C-NMR: δ0.45(SiMe₃), 9.5(CH₃), 34.8(CH₂), 40.4(N(CH₃)₂), 69.2(C), 92.2, 103.7(C≡C), 127.0, 127.4, 127.8, 142.3(Ar).

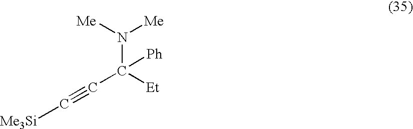

wherein Me represents methyl group, Ph represents phenyl group, and Et represents ethyl group.

### EXAMPLE 23

Diethyl ether (5 ml) was placed in a 50 ml two-necked flask that had been subjected to reduced-pressure drying and argon substitution. Thereafter, 0.22 ml (1.5 mmol) of propargyl aldehyde diethyl acetal and 0.94 ml (1.6 M solution in hexane; 1.5 mmol) of butyl lithium were added thereto at a temperature of 0° C. , followed by stirring for 10 minutes, so as to obtain lithium acetylide. This reaction solution was defined as solution M.

Using an L-shaped tube, solution M was added to solution L of Example 22 that had been cooled to 0° C. , followed by stirring at 20° C. for 30 minutes. Subsequently, 10 ml (1.0 M solution in Et₂O; 10 mmol) of trimethylsilylmethyl magnesium chloride was added to the obtained reaction solution, followed by stirring at 42° C. for 6 hours. Thereafter, 20 ml of saturated ammonium chloride aqueous solution was further added thereto, and the reaction was then terminated. Thereafter, ether extraction was repeatedly carried out 3 times on the thus obtained reaction solution, and extraction of the ether layer was repeatedly carried out 3 times thereon using 6 ml of concentrated hydrochloric acid. Subsequently, the obtained extract was adjusted to alkaline pH (pH=13 to 14) with a 30% sodium hydroxide aqueous solution, and ether extraction was repeatedly carried out 5 times thereon using 6 ml of diethyl ether. Further, extraction of the ether layer was repeatedly carried out 3 times thereon using 6 ml of concentrated hydrochloric acid. The obtained extract was adjusted to alkaline pH (pH=13 to 14) with a 30% sodium hydroxide aqueous solution, and ether extraction was repeatedly carried out 5 times thereon using 6 ml of diethyl ether. Thereafter, the extract was dried with anhydrous magnesium sulfate, followed by filtration and concentration, so as to obtain dark brown oil. The yield of the dark brown oil was 0.205 g (75% yield). From the results of infrared absorption spectrometry and nuclear magnetic resonance spectrometry, it was found that the dark brown oil of Example 23 was N,N-dimethyl-α-(2-formylethynyl)-α-[(1-trimethylsilyl)methyl]-benzenemethanamine represented by structural formula (36).

<IR (KBr disk)> (neat)3060, 3027, 2952, 2921, 2865, 2825, 2782, 2207, 1668, 1448, 1247, 857, 837 cm⁻¹

<NMR (in CDCl₃, TMS internal standard)>

¹H-NMR: δ-0.32(s, 9H, SiMe₃), 1.36(d, J=14.2 Hz, 1H, CH₂), 1.65(d, J=14.2 Hz, 1H, CH₂), 2.21(s, 6H, N(CH₃)₂), 7.26-7.36(m, 3H, Ar), 7.58-7.60(m, 2H, Ar), 9.42(s, 1H, CHO).

¹³C-NMR: δ-0.82(SiMe₃), 32.1(CH₂), 40.0(N(CH₃)₂), 66.0(C), 88.7, 96.8(C≡C), 126.7, 127.9, 128.4, 142.1(Ar), 176.7(CHO).

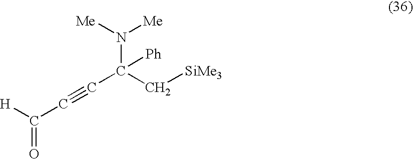

wherein Me represents methyl group, and Ph represents phenyl group.

### EXAMPLE 24

Diethyl ether (5 ml) was placed in a 20 ml two-necked flask that had been subjected to reduced-pressure drying and argon substitution. Thereafter, 0.14 ml (1.5 mmol) of 2-methyl-1-buten-3-yne and 0.94 ml (1.6 M solution in hexane; 1.5 mmol) of butyl lithium were added thereto at a temperature of 0° C., followed by stirring for 10 minutes, so as to obtain lithium acetylide. This reaction solution was defined as solution N. At the same time, 5 ml of diethyl ether, 0.244 g (1 mmol) of N,N-dimethyl-4-bromobenzenecarbothioamide, and 0.115 ml (1 mmol) of methyl trifluoromethanesulfonate were successively placed in a 50 ml two-necked flask that had been subjected to reduced-pressure drying and argon substitution, followed by stirring at 20° C. for 30 seconds. This reaction solution was defined as solution O.

Solution N was added to solution 0 that had been cooled to 0° C., using an L-shaped tube, followed by stirring at 20° C. for 30 minutes. Subsequently, 10 ml (1.0 M solution in Et₂O; 10 mmol) of allyl magnesium bromide was added to the obtained reaction solution, followed by stirring at 20° C. for 6 hours. Thereafter, 20 ml of saturated ammonium chloride aqueous solution was further added thereto, and the reaction was then terminated. Thereafter, ether extraction was repeatedly carried out 3 times on the thus obtained reaction solution, and extraction of the ether layer was repeatedly carried out 3 times thereon using 6 ml of concentrated hydrochloric acid. Subsequently, the obtained extract was adjusted to alkaline pH (pH=13 to 14) with a 30% sodium hydroxide aqueous solution, and ether extraction was repeatedly carried out 5 times thereon using 6 ml of diethyl ether. Further, extraction of the ether layer was repeatedly carried out 3 times thereon using 6 ml of concentrated hydrochloric acid. The obtained extract was adjusted to alkaline pH (pH=13 to 14) with a 30% sodium hydroxide aqueous solution, and ether extraction was repeatedly carried out 5 times thereon using 6 ml of diethyl ether. Thereafter, the extract was dried with anhydrous magnesium sulfate, followed by filtration and concentration, so as to obtain light yellow oil. The yield of the light yellow oil was 0.224 g (70% yield). From the results of infrared absorption spectrometry and nuclear magnetic resonance spectrometry, it was found that the light yellow oil of Example 24 was N,N-dimethyl-α-(3-methyl-3-buten-1-ynyl)-α-(2-propenyl)-4-bromobenzene methaneamine represented by structural formula (37).

<IR (KBr disk)> (neat)3077, 2982, 2952, 2919, 2864, 2825, 2783, 1614, 1586, 1484, 1291, 1011, 822 cm⁻¹

<NMR (in CDCl₃, TMS internal standard)>

¹H-NMR: δ1.98(s, 3H, CH₃), 2.20(s, 6H, N(CH₃)₂), 2.57(dd, J=7.6, 13.4 Hz, 1H, CH₂ having a single bond with CH of CH₂═CHCH₂), 2.74(dd, J=7.6, 13.4 Hz, 1H, CH₂ having a single bond with CH of CH₂═CHCH₂), 4.83-4.89(m, 2H, CH₂ having a double bond with CH of CH₂═CHCH₂), 5.26-5.28(m, 1H, CH₂═C), 5.37-5.48(m, 2H, CH₂ in CH₂═C, and CH in CH₂═CHCH₂), 7.42(d, J=8.8 Hz, 2H, Ar), 7.48(d, J=8.8 Hz, 2H, Ar).

¹³C-NMR: δ24.0(CH₃), 40.4(NMe₂), 46.8(CH₂ having a single bond with CH of CH₂═CHCH₂), 67.6(C), 85.4, 90.3(C≡C), 118.0(CH₂ having a double bond with CH of CH₂═CHCH₂), 121.0(C in C═CH₂), 121.6(CH₂ in C═CH₂), 126.6, 129.3, 130.9(Ar), 133.3(CH in CH₂═CHCH₂), 141.8(Ar).

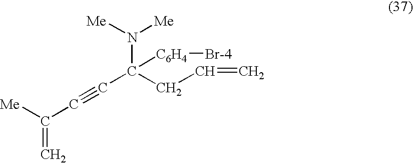

wherein Me represent methyl group, and C₆H₄-Br-4 represents 4-bromophenyl group.

The present examples and embodiments are to be considered as illustrative and not restrictive, and the invention is not to be limited to the details given herein, but may be modified within the scope and equivalence of the appended claims.

What is claimed is:

1. A tertiary amine represented by the following general formula (1): 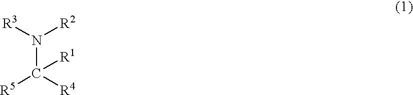
wherein R¹ represents hydrogen atom, alkyl group or aryl group; R² and R³ each represent alkyl group or allyl group; R⁴ represents alkyl group, aryl group or allyl group; R⁵ represents alkynyl group, aryl group or alkyl group; and wherein when R⁵ is aryl or alkyl group, R¹, R⁴ and R⁵ are different from one another.

2. The tertiary amine according to claim 1, which is a propargylamine represented by the following general formula (2): 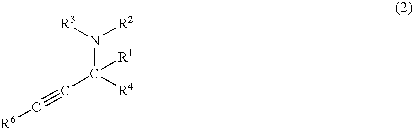
wherein R¹ represents hydrogen atom, alkyl group or aryl group; R² and R³ each represent alkyl group or allyl group; R⁴ represents alkyl group, aryl group or allyl group; and R⁶ represents alkyl group, aryl group, silyl group, vinyl group or formyl group having 2 or more carbon atoms.

3. A method for producing a tertiary amine represented by the following general formula (3): 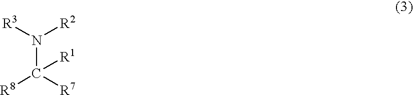
wherein R¹ represents hydrogen atom, alkyl group or aryl group; R²and R³ each represent alkyl group or allyl group; R⁷ represents alkyl group, aryl group, allyl group, vinyl group or alkynyl group; and R⁸ represents alkynyl group, aryl group or alkyl group, the production method comprising the steps of:
adding thioamide represented by general formula (4) and a methylating agent represented by general formula (5) into a solvent,
adding thereto a metal-containing reagent represented by general formula (6), and
adding thereto a Grignard reagent represented by general formula (7), 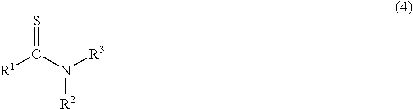
wherein R¹ represents hydrogen atom, alkyl group or aryl group, and R² and R³ each represent alkyl group or allyl group,
CH₃—X (5)
wherein X represents perfluoroalkyl sulfoxyl group,
R⁸-M¹ (6)
wherein R⁸ represents alkynyl group, aryl group or alkyl group, and M¹ represents an alkali metal atom, and
R⁷-M² (7)
wherein R⁷ represents alkyl group, aryl group, allyl group, vinyl group or alkynyl group, and M² represents MgCl, MgBr or MgI.

4. The production method according to claim 3, wherein said metal-containing reagent is represented by the following general formula (8):
R⁶—C≡C-M¹ (8)
wherein R⁶ represents alkyl group, aryl group, silyl group, vinyl group or dialkoxymethyl group having 2 or more carbon atoms, and M¹ represents an alkali metal atom.

5. The production method according to claim 3, wherein said step of adding a Grignard reagent is carried out at a temperature between 40° C. and 70° C. when said R¹ represents aryl or alkyl group, and the same above step is carried out at a temperature between 0° C. and 35° C. when said R¹ represents hydrogen atom.

6. The production method according to claim 3, wherein said solvent is diethyl ether or tetrahydrofuran.

7. The production method according to claim 3, wherein said step of adding a metal-containing reagent comprises mixing a solvent containing said thioamide and said methylating agent with a solvent containing said metal-containing reagent.

8. The production method according to claim 3, further comprising the steps of:
stirring the solvent for at least 15 minutes after the step of adding said thioamide and said methylating agent thereto,
stirring the solvent for at least 15 minutes after the step of adding said metal-containing reagent, and
stirring the solvent for at least 15 minutes after the step of adding said Grignard reagent thereto.

9. A method for producing a tertiary amine represented by the following general formula (3): 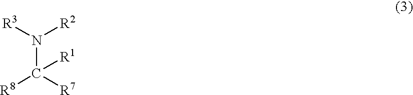
wherein R¹ represents hydrogen atom, alkyl group or aryl group; R² and R³ each represent alkyl group or allyl group; R⁷ represents alkyl group, aryl group, allyl group, vinyl group or alkynyl group; and R⁸ represents alkynyl group, aryl group or alkyl group, the method comprising the steps of:
adding a metal-containing reagent represented by general formula (6) into a reaction system including a solvent, thioamide represented by general formula (4), and a methylating agent represented by general formula (5); and then
adding thereto a Grignard reagent represented by general formula (7), 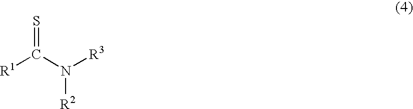
wherein R¹ represents hydrogen atom, alkyl group or aryl group, and R² and R³ each represent alkyl group or allyl group,
CH₃—X (5)
wherein X represents perfluoroalkyl sulfoxyl group,
R⁸-M¹ (6)
wherein R⁸ represents alkynyl group, aryl group or alkyl group, and M¹ represents an alkali metal atom, and
R⁷-M² (7)
wherein R⁷ represents alkyl group, aryl group, allyl group, vinyl group or alkynyl group, and M² represents MgCl, MgBr or MgI, wherein the equivalent ratio of said thioamide:said methylating agent:said metal-containing reagent:said Grignard reagent is within the range of 1:1:(1.2 to 1.5):(1.5 to 10).

10. The production method according to claim 9, wherein said R¹ represents hydrogen atom, and the temperature of said reaction system is between 0° C. and 35° C. when said Grignard reagent is added.

11. The production method according to claim 9, wherein said R¹ represents aryl or alkyl group, and the temperature of said reaction system is between 40° C. and 70° C. when said Grignard reagent is added.

12. The production method according to claim 9, wherein
said step of adding said metal-containing reagent includes subjecting to an addition reaction a reaction intermediate between said thioamide and said methylating agent, and said metal-containing reagent so as to generate a first addition product, and
said step of adding said Grignard reagent includes subjecting to an addition reaction said first addition product and said Grignard reagent so as to generate a second addition product.
